(19) United States
(12) Patent Application Publication
(10) Pub. No.: US 2012/0271180 A1
(43) Pub. Date: Oct. 25, 2012

(54) MULTIFUNCTIONAL MOUSE

(75) Inventors: FAN REN, Shenzhen City (CN); YUE-HUA SHEN, Shenzhen City (CN)

(73) Assignees: HON HAI PRECISION INDUSTRY CO., LTD., Tu-Cheng (TW); HONG FU JIN PRECISION INDUSTRY (ShenZhen) CO., LTD., Shenzhen City (CN)

(21) Appl. No.: 13/177,559

(22) Filed: Jul. 7, 2011

(30) Foreign Application Priority Data
Apr. 22, 2011 (CN) 201110101743.4

(51) Int. Cl. A61B 5/02; A61B 5/00; A61B 5/01

(52) U.S. Cl. 600/500; 600/549; 600/300

(57) ABSTRACT

A mouse includes an aperture defined in its top, a light source, and a data processing unit partially positioned external to the housing from the aperture. When the mouse is covered by a palm of a user and the light source emits infrared light, the infrared light reaches the palm of the user through the aperture, and some of the infrared light is reflected and received by the data processing unit. The data processing unit calculates and measures the heart rate and body temperature of the user against physiological parameters.

### BACKGROUND

1. Technical Field

The present disclosure relates to computer mice, and particularly to a multifunctional mouse.

2. Description of Related Art

If a person uses a computer for a long time, the health of the person may be adversely affected by frequent and long-term operations on the computer, the wrong posture in operating the computer, and the deleterious effects of electromagnetic waves emitted by the computer. Many physiological parameters of the user, such as his/her heart rate and body temperature, may undergo abnormal change, but the user is not aware of these changes in time to avoid or prevent them.

Therefore, there is room for improvement within the art.

### BRIEF DESCRIPTION OF THE DRAWINGS

Many aspects of the present disclosure can be better understood with reference to the following drawings. The components in the various drawings are not necessarily drawn to scale, the emphasis instead being placed upon clearly illustrating the principles of the present disclosure. Moreover, in the drawings, like reference numerals designate corresponding parts throughout the figures.

FIG. 1 is a schematic view of a mouse, according to an exemplary embodiment.

FIG. 2 is a schematic view of the mouse shown in FIG. 1 in use.

FIG. 3 is a block diagram of the electronic connections between a data processing unit, a display unit, and an alarm unit of the mouse shown in FIG. 1.

### DETAILED DESCRIPTION

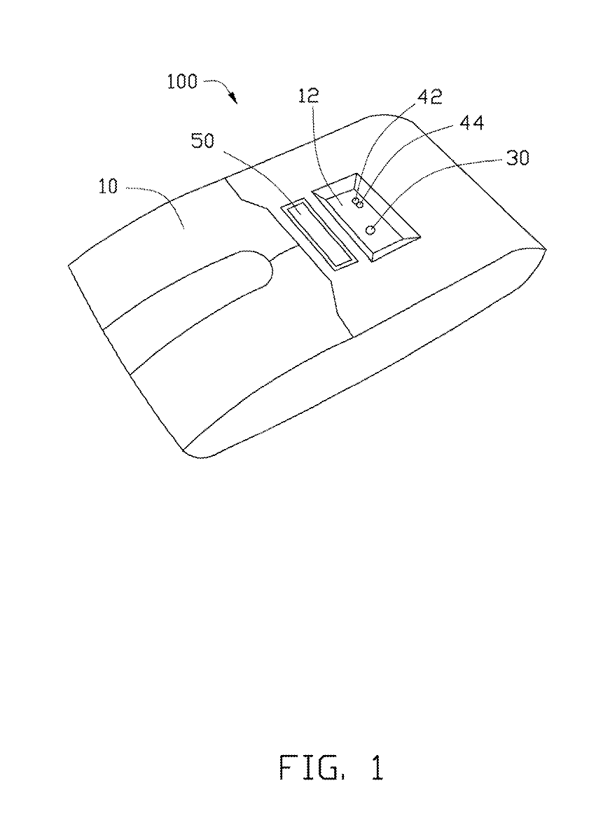

FIG. 1 schematically shows a mouse 100 according to an exemplary embodiment. The mouse 100 can be used to operate computers as a typical mouse, and can also be used to measure certain physiological parameters of users, such as their heart rates and body temperatures.

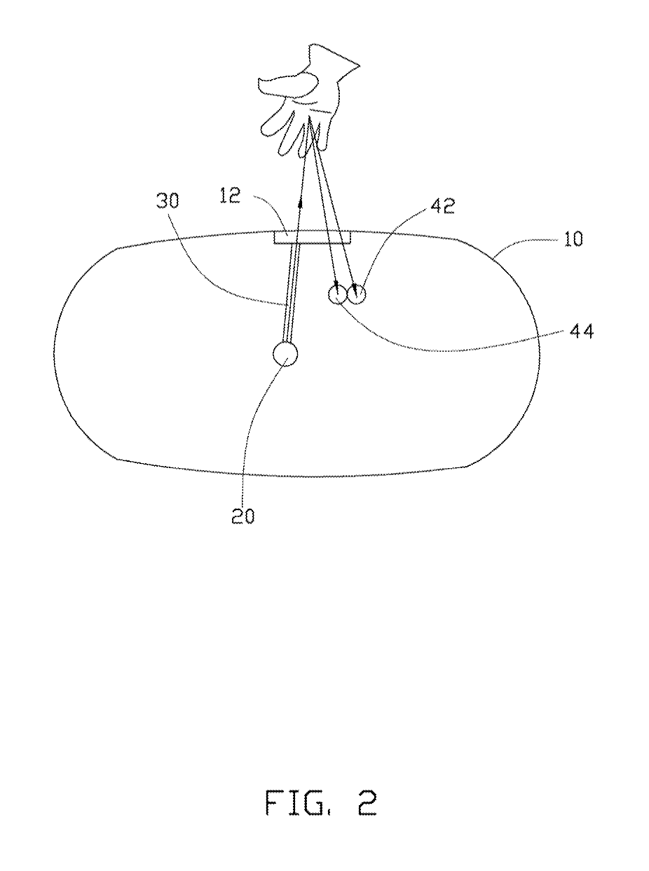

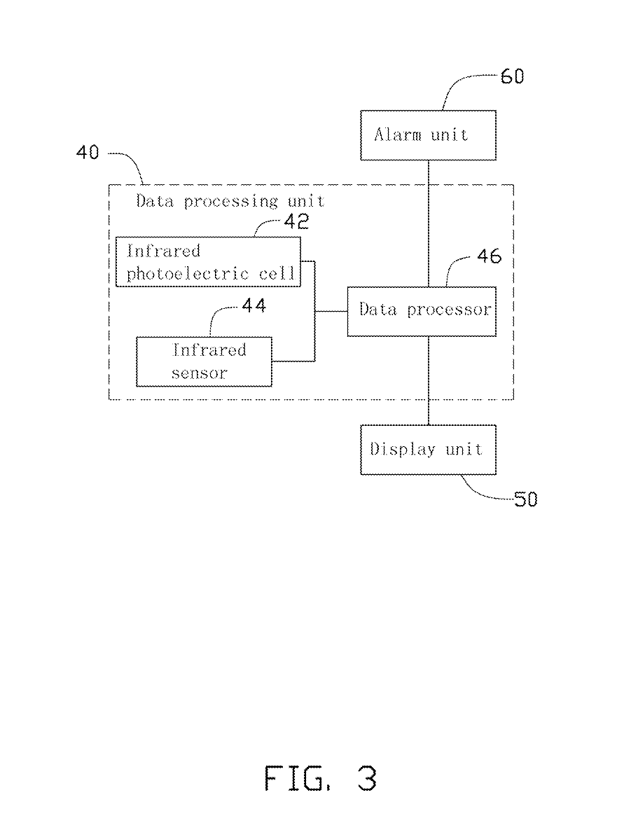

Also referring to FIG. 2 and FIG. 3, the mouse 100 includes a housing 10, a light source 20, a light guide 30, a data processing unit 40, a display unit 50, and an alarm unit 60. The light source 20, the light guide 30, the data processing unit 40, the display unit 50, and the alarm unit 60 are all received in the housing 10. Furthermore, the mouse 100 includes all the necessary components (not shown) of common computer mice as will be know of one of ordinary skill in the art would know.

The housing 10 defines an aperture 12 in its top part. When a user grasps and operates the mouse 100, the aperture 12 is intended to be aligned with a palm of the user. The light source 20 is an infrared light source that can be independent or integrated with a typical infrared light source for photoelectric mice. The light guide 30 has one end positioned in a light emitting path of the light source 20 to receive infrared light emitted by the light source 20, and another end positioned external to the housing 10 from the aperture 12. In this way, when the mouse 100 is used, the light guide 30 can guide the infrared light emitted by the light source 20 to the aperture 12, such that the infrared light can reach a palm of the user through the aperture 12.

The data processing unit 40 includes a infrared photoelectric cell 42, an infrared sensor 44, and a data processor 46. Both the infrared photoelectric cell 42 and the infrared sensor 44 are electrically connected to the data processor 46 and positioned external to the housing 10 from the aperture 12. When the infrared light emitted by the light source 20 reaches the palm of the user, some of the infrared light is reflected back by the palm of the user and received by the infrared photoelectric cell 42 and the infrared sensor 44.

The display unit 50 is electrically connected to the data processor 46 and positioned external to the housing from a top surface of the housing 10. The display unit 50 can be a typical liquid crystal display (LCD) or a typical seven segment light emitting diode (LED) display, etc. The data processor 46 can control the display unit 50 to display characters and images. The alarm unit 60 is electrically connected to the data processor 46. The alarm unit 60 can be a buzzer, a speaker, a vibrator, or a combination of these components. The data processor 46 can control the alarm unit 60 to generate sound signals and/or vibrations.

In use, the light source 20 emits infrared light. The light guide 30 guides the infrared light emitted by the light source 20 to the aperture 12, such that the infrared light emits out of the housing 10 through the aperture 12. When the mouse 100 is grasped and operated in a palm of a user, the infrared light reaches the palm of the user through the aperture 12. Additionally, if the light source 20 is integrated with a typical infrared light source for photoelectric mice, the infrared light emitted by the light source 20 can also be used to identify lateral movements of the mouse 100 and further to operate the computer (not shown) connected to the mouse 100, by such means as would be known to those of ordinary skill in the art.

When the infrared light emitted by the light source 20 reaches the palm of the user, some of the infrared light is reflected by the palm of the user and received by the infrared photoelectric cell 42 and the infrared sensor 44. When the heart of the user rhythmically contracts to drive blood around the body of the user, a transparency of the palm of the user decreases, and the brightness of the infrared light reflected by the palm of the user increases. When the heart muscle relaxes between rhythmic contractions, blood returns to the heart, a transparency of the palm of the user increases, and the brightness of the infrared light reflected by the palm of the user decreases. Accordingly, the data processor 46 continuously measures a brightness of the infrared light received by the infrared photoelectric cell 42, and calculates a heart rate of the user according to the number of cycles per minute of the increases and decreases in the brightness of the infrared light received by the infrared photoelectric cell 42. Thus, the data processor 46 controls the display unit 50 to display the heart rate of the user. If the heart rate of the user is outside a predetermined range, the data processor 46 identifies an unhealthy situation, and controls the alarm unit 60 to generate sound signals and/or vibrations, thereby reminding the user to rest.

Furthermore, using the infrared sensor 44, the data processor 46 obtains an infrared image of a part of the palm of the user that reflects infrared light to the infrared sensor 44, and measures the temperature of the palm of the user from the infrared image. On the basis that the infrared image is the sum of the infrared light emitted by the light source 20 and reflected to the infrared sensor 44, and the infrared light inherently emitted by the palm of the user and transmitted to the infrared sensor 44, the temperature of a palm of the user can be established. When a body temperature of the user changes, the intensity or strength of the infrared light emitted by the palm of the user changes, and the color and/or brightness of the infrared image obtained by the data processor 46 correspondingly changes. The data processor 46 calculates the body temperature(s) of the user according to changes in the color and/or brightness of the infrared image. If the body temperature change of the user is outside a predetermined range, the data processor 46 identifies that the user is in an unhealthy state, and controls the alarm unit 60 to generate sound signals and/or vibrations, thereby reminding the user to rest.

Additionally, when the light source 20 is turned off and the palm of the user is aligned with the aperture 12, the infrared sensor 44 can only receive the infrared light inherently emitted by the palm of the user. In this way, the data processor 46 obtains an infrared image of a part of the palm of the user which is aligned with the infrared sensor 44 using the infrared sensor 44, comprising only the infrared light emitted by the palm of the user. Thus, the data processor 46 can measure and record a body temperature of the user according to such an infrared image, and control the display unit 50 to display the body temperature of the user.

The present disclosure can measure and display the heart rates and changes in body temperature of users when serving as a typical mouse. When the heart rates and body temperature changes of the users are outside predetermined ranges (i.e., the heart rates and/or body temperatures of the users change excessively or reach abnormal levels), the data processor 46 can control the alarm unit 60 to remind the users to rest in time.

It is to be further understood that even though numerous characteristics and advantages of the present embodiments have been set forth in the foregoing description, together with details of structures and functions of various embodiments, the disclosure is illustrative only, and changes may be made in detail, especially in matters of shape, size, and arrangement of parts within the principles of the present invention to the full extent indicated by the broad general meaning of the terms in which the appended claims are expressed.

What is claimed is:

1. A mouse, comprising:
a housing defining an aperture;
a light resource received in the housing; and
a data processing unit received in the housing and partially positioned external to the housing from the aperture; wherein the light source emits infrared light through the aperture, and reflected infrared light is received and measured by the data processing unit.

2. The mouse as claimed in claim 1, wherein the reflected infrared light is reflected by a hand of a user contacting the mouse.

3. The mouse as claimed in claim 2, wherein the measured reflected infrared light is used to evaluate physiological parameters of the user.

4. The mouse as claimed in claim 3, wherein the data processing unit includes a data processor and an infrared photoelectric cell, the infrared photoelectric cell is connected to the data processor and positioned external to the housing from the aperture for receiving the reflected infrared light.

5. The mouse as claimed in claim 4, wherein the data processor measures a brightness of the reflected infrared light received by the infrared photoelectric cell, and calculates a heart rate of the user according to increasing/decreasing periods of the brightness of the reflected infrared light received by the infrared photoelectric cell.

6. The mouse as claimed in claim 5, wherein the infrared light emitted by the light source is further used to identify movements of the mouse.

7. The mouse as claimed in claim 5, wherein the data processing unit further includes an infrared sensor connected to the data processor and positioned external to the housing from the aperture for receiving the reflected infrared light; when the infrared sensor receives the reflected infrared light, the data processor obtains an infrared image of a part of the palm of the user using the infrared sensor, and measures a temperature of the palm of the user according to the infrared image.

8. The mouse as claimed in claim 7, wherein the data processor calculates a body temperature change of the user according to the color and/or brightness changes of the infrared image.

9. The mouse as claimed in claim 7, wherein when the light source is turned off and the infrared sensor receives the infrared light emitted by the palm of the user, the data processor obtains an infrared image of a part of the palm of the user using the infrared sensor, and measures a body temperature of the user according to the infrared image.

10. The mouse as claimed in claim 3, further comprising a display unit connected to the data processing unit and positioned external to the housing from a surface of the housing;
wherein the data processing unit controls the display unit to display data of the measured physiological parameters of the user.

11. The mouse as claimed in claim 3, further comprising an alarm unit received in the housing and connected to the data processing unit; wherein the data processing unit controls the alarm unit to alarm when the measured physiological parameters of the user are outside predetermined ranges.
